(19) United States
(12) Patent Application Publication
(10) Pub. No.: US 2012/0285987 A1
(43) Pub. Date: Nov. 15, 2012

(54) POWDER DISPENSER WITH CARTRIDGE

(76) Inventor: John LEIER, San Diego, CA (US)

(21) Appl. No.: 13/467,926

(22) Filed: May 9, 2012

Related U.S. Application Data
(60) Provisional application No. 61/484,097, filed on May 9, 2011.

(51) Int. Cl. A61M 35/00; B05C 17/01

(52) U.S. Cl. 222/1; 222/162

(57) ABSTRACT

Described are articles that serve as integrated dispensers or applicators for powdered compositions. Primarily, the powder-dispensing articles of the invention include a housing in which a cartridge is disposed that can move in relation to the housing, typically by depressing a plunger that protrudes from the housing. The housing unit contains a housing access port that provides access to the preferably replaceable cartridge. The cartridge also contains an orifice, referred to as a cartridge access port, in the cartridge wall. The housing and cartridge access ports are brought into alignment by the user, for example, by depressing a plunger that causes the cartridge to move in relation to the housing by compressing a biasing member. Such port alignment exposes the powdered composition inside the cartridge.

### 1. FIELD OF THE INVENTION

The present invention relates to articles for dispensing powdered compositions, and methods of using such articles.

The following description includes information that may be useful in understanding the present invention. It is not an admission that any of the information provided herein, or any publication specifically or implicitly referenced herein, is prior art, or even particularly relevant, to the presently claimed invention.

### 2. BACKGROUND

Veterinarians, technicians, groomers and even some pet owners often find themselves applying styptic powder to bleeding toe nails after cutting the nails too short. “Cutting to the quick” is a common expression in the pet care profession, and refers to cutting into the blood supply of the affected nail. There are various products on the market to expedite the clotting of blood, including gel and silver nitrate sticks, although by far the most commonly used topical hemostatic agent for this purpose is a styptic powder available through various vendors, all with varying methods of application. While the most prominent and widely used method of stopping blood flow from a bleeding toe nail has been the use of a styptic powder, its application is crude at best, and is often performed by manual application of loose powder first placed on a person's finger tip and then applied directly to the animal's nail or, alternatively, by applying a cotton tip applicator loaded with styptic powder directly to the nail. In either of these approaches, the user often fumbles with the styptic powder while the animal struggles, with the result often being the spillage of the styptic powder, either because of user error or the bleeding animal knocking the powder from the user's hand. Either of such outcomes results in ineffective application and loss of product. In addition to being wasteful, conventional approaches are time consuming. Moreover, in conventional approaches the styptic powder is frequently overexposed to the atmosphere or to air and humidity, the result of which is caking of the powder.

While various applicators have been developed as attempts to improve delivery of styptic powders, the need still exists for a simple, inexpensive hand-held device that a user can employ to efficiently and effectively administer a hemostatic amount of a styptic powder (or other composition capable of stemming blood flow) to a bleeding toenail.

### 3. DEFINITIONS

Before describing the instant invention in detail, several terms used in the context of the present invention will be defined. In addition to these terms, others are defined elsewhere in the specification as necessary. Unless otherwise expressly defined herein, terms of art used in this specification will have their art-recognized meanings.

An “antihemorrhagic” or “hemostatic” compound or composition refers to a substance or composition that promotes hemostasis, i.e., stops bleeding. A “styptic” compound or composition refers to a class of antiseptic hemostatic substances or compositions that function by stimulating contraction of tissue, particularly blood vessels. An example of a commercially available styptic powder is KwicStop® (Arc Laboratories, Inc., Atlanta, Ga.: a hemostatic composition that includes ferric subsulfate, aluminum chloride, diatomite bentonite, copper sulfate, ammonium chloride, idophor, and benzocaine). Other topical hemostatic substances or compositions that can also be used in practicing the invention include coagulants, i.e., compounds and compositions that promote platelet aggregation, such as microfibrillar collagen and chitosan.

A “patentable” article of manufacture, device, machine, process, or composition of matter according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the non-patentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

A “plurality” means more than one.

### 4. SUMMARY OF THE INVENTION

Thus, one object of the invention relates to articles adapted to deliver a powered composition, for example, a styptic powder. The powder dispensers of the invention house replaceable powder filled-cartridges that are easy to use. In some embodiments, such articles can be used for applying styptic powder to a bleeding animal's toenail. In other embodiments, they are used dispensing an approximate measure of different powdered compositions, for example, a cooking spice.

In general, the powder-dispensing articles of the invention include a housing that houses a cartridge that can move in relation to the housing, typically by depressing a plunger that protrudes from the housing. After use, the cartridge preferably returns to an idle position. In most embodiments, the housing is an elongated housing that has an open end opposite a sealed end and a housing access port disposed proximal to the sealed end so as to provide access to the interior of the housing. The cartridge is disposed inside of and in moveable relation to the housing, and comprises first and second sealed ends disposed opposite each other so as to define a reservoir configured to store a powdered composition. Cartridges also include a cartridge access port that provides access to the reservoir, particularly to powdered contents stored in the reservoir. Preferably, the first sealed end of a cartridge faces the sealed end of the housing in which the cartridge is placed, while the cartridge's second sealed end faces the open end of the housing. The plunger comprises a plunger element that has a flange disposed at one end and at the other end, an exposed surface configured for application of an actuation force by a user.

The flange has a cartridge-facing surface that engages the second sealed end of the cartridge and a retaining surface opposite the cartridge-facing surface. The plunger element extends through the open end of the housing and is retained in the housing by a plunger retainer disposed on the open end of the housing. The dispensing articles of the invention also preferably include a biasing member (e.g., a spring) disposed inside the housing and positioned to engage and return the cartridge to a resting or “idle” position after an actuation force applied by a user is reduced or removed. As will be appreciated, application of an actuation force by a user will causes the cartridge to move in relation to the housing and bring the cartridge and housing access ports into alignment, thereby allowing access to the reservoir

As those in will appreciate, a clear advantage afforded by the articles of the invention is that the replaceable cartridge within the housing is kept semi-airtight and spill resistant, and thereby minimizes waste of the powdered composition contained within the cartridge. Also, the cartridges preferably contain quantities of the desired composition to enable multiple independent uses of the device, thereby avoiding a requirement for manual filling for a given application. Moreover, because the powdered composition is stored safely within the cartridge, users can avoid contact with the cartridge contents. As opposed to the currently available styptic powder dispensers, spice dispensers, and other powdered composition dispensers, which are primarily storage containers for loose powdered compositions, the powdered compositions within a cartridge of an article according to the invention is less subject to spillage and atmospheric conditions.

The articles of the invention can also further include one or more other components, including any one or more of an alignment system that provides complementary structures between the housing and cartridge to promotes alignment between the housing access port and cartridge access port when a cartridge is moved into dispensing position, a stop disposed in the reservoir opposite the cartridge access port, and a powdered composition in the reservoir.

Another aspect of the invention concerns methods of dispensing a powdered composition from a cartridge in an article according to the invention. Typically, this is accomplished by a user applying an actuation force to the article's plunger to bring the housing access port and cartridge access port into alignment to allow at least a portion of the powdered contents within the cartridge to be dispensed.

These and other aspects and embodiments of the invention are discussed in greater detail in the sections that follow. The foregoing and other aspects of the invention will become more apparent from the following detailed description, accompanying drawings, and the claims. Although methods and materials similar or equivalent to those described herein can be used in the practice or testing of the present invention, suitable methods and materials are described below. In addition, the materials, methods, and examples below are illustrative only and not intended to be limiting.

### 5. BRIEF DESCRIPTION OF THE DRAWINGS

A brief summary of each of the figures is provided below.

FIG. 1 shows a representative embodiment of a device of the invention from a top perspective view.

FIG. 2 shows the device depicted in FIG. 1 in an exploded perspective view.

FIG. 3 shows an inner cartridge of a device according to the invention from a side elevational view.

FIG. 4 shows a representative embodiment of a device of the invention from an exploded perspective view.

FIG. 5 shows another representative embodiment of a device from an exploded perspective view.

FIG. 6 shows the inner cartridge in its entirety from a side elevational view.

In the drawings, the following reference numerals correspond to the indicated component, unless otherwise indicated.

Drawings - Reference Numerals

| | |
|---|---|
| 22 Plunger Retainer | 24 Plunger cover |
| 26 Plunger | 28 Cartridge |
| 30 Square cut o-ring | 31 Tongue |
| 32 Saddle seal (bearing) | 34 Cartridge access port |
| 36 Cartridge bottom plug | 38 Biasing Member |
| 40 Outer housing tube | 42 Stabilizing notch |
| 44 Housing access port | 46 Sliding sleeve |
| 48 Housing bottom plug | 50 Nail stop point |
| 52 Pocket clip (optional) | 54 Magnet (optional) |
| 49 Saddle seal (bearing) | |

### DETAILED DESCRIPTION

The following description describes several representative embodiments of certain preferred dispensing articles of the invention. These embodiments are non-limiting.

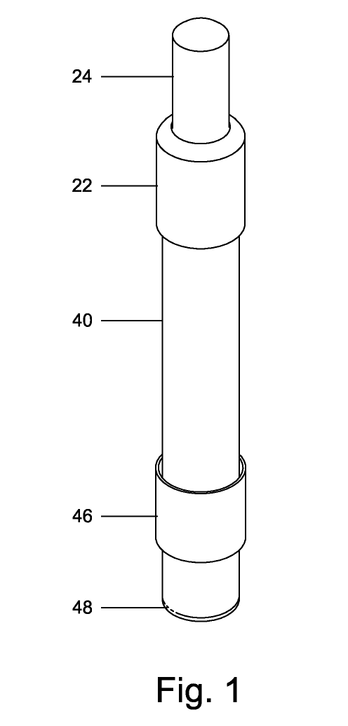

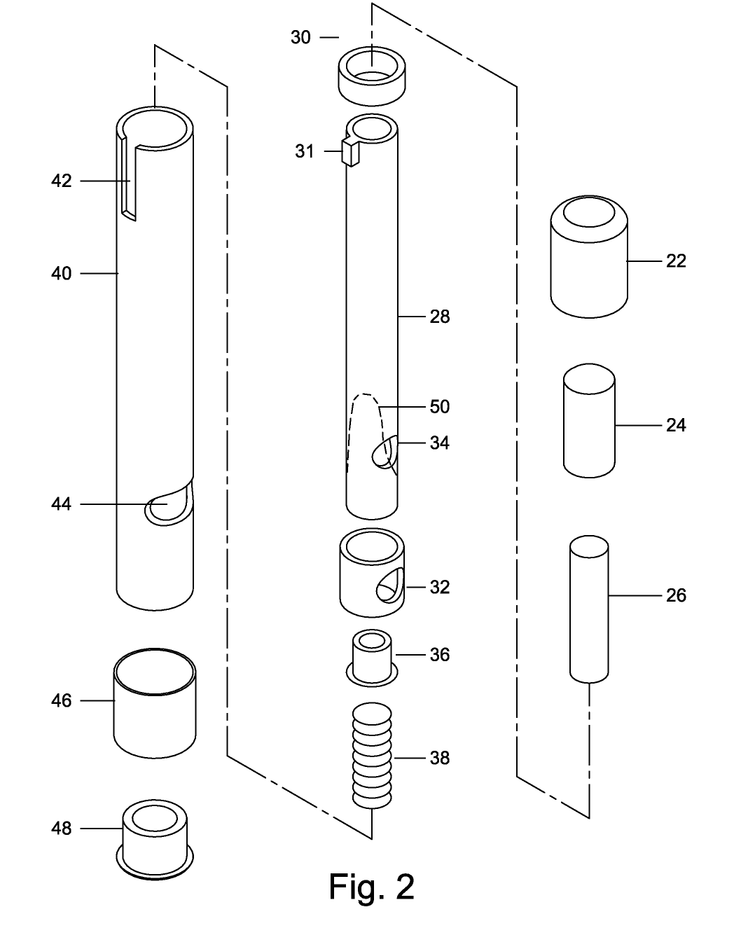

A first embodiment is illustrated in FIG. 1. The powder dispenser includes an outer housing tube 40, which in this embodiment is made of 0.75 in. anodized aluminum tubing or other workable materials such as cardboard, stainless steel, or polycarbonate plastic. The outer housing can be made in various lengths, with 4-7 in. being preferred, and a length of about 5.5 in. being particularly preferred. The housing 40 is topped with a replaceable 0.75 in. plunger retainer 22 that has a 0.5 in. hole bored out of the center. The plunger retainer 22 can be made of any suitable material, including vinyl, plastic, or threaded metal. The 0.5 in. plunger cover 24 is also made of vinyl or a polycarbonate plastic, and protrudes from the inner cartridge (not shown in FIG. 1) and conceals the plunger 26 (shown FIG. 2) made of acrylic, polycarbonate plastic, or other suitable materials. The housing 40 also includes a sliding sleeve 46, which can made of vinyl or polycarbonate plastic. The bottom of the housing 40, opposite the end from which the plunger protrudes (i.e., the top end of the housing 40), is sealed. In this embodiment, the bottom end of housing 40 is sealed, for example, with a bottom plug 48, made of polycarbonate plastic. The bottom plug 48 may be permanently attached or be removable. In some embodiments, the bottom of the housing may be sealed, for example, by crimping the housing and eliminating the bottom plug 48, while in still other embodiments, the bottom of the housing is left open.

FIG. 2

Starting at the top down is a plunger retainer 22 that slides over the top of the plunger cover 24 and the plunger 26 finally resting on top of the outer housing tube 40. The plunger 26 is concealed by the plunger cover 24 and both the plunger 26 and plunger cover 24 are attached to the top of the cartridge 28. The cartridge 28 carries a square cut o-ring 30 that rests just below the tongue 31. The lower portion of the cartridge is saddled with a saddle seal 32, which is centered over the cartridge access port 34.

The entire cartridge 28 is sealed on the bottom with a cartridge bottom plug 36, which be made of polycarbonate plastic. A biasing member 38 is positioned inside of the outer housing tube 40 resting at the bottom on top of the inner housing bottom plug 48.

A ⅞″ deep stabilizing notch 42 is milled from the top wall of the outer housing tube 40. The stabilizing notch serves as a groove for the tongue 31 to slide in. The housing access port 44 is bored out about 1″ from the bottom of the outer housing tube 40. The sliding sleeve 46 rides on the outer wall of the outer housing tube 40 and is placed atop of the housing access port 44 concealing it while in its resting position. The outer housing tube 40 is sealed on the bottom with the housing bottom plug 48. An optional magnet 54 may be attached to the inner wall of the outer housing tube 40.

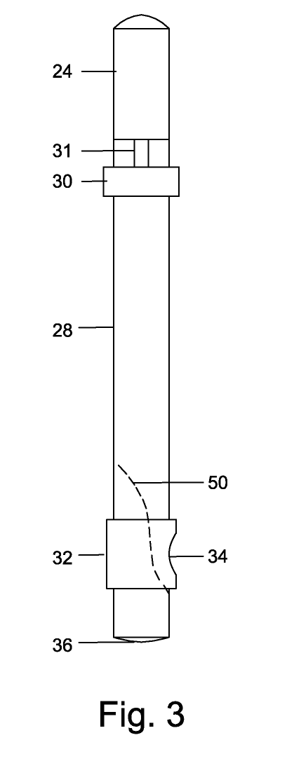

FIG. 3

The plunger cover 24 conceals the plunger 26, which is bonded to the inner wall of the cartridge 28 sealing in a powdered composition. The square cut o-ring 30 wrapped around the cartridge 28 rests below the tongue 31 and the saddle seal 32 is positioned over the cartridge access port 34 within the cartridge 28 just below the cartridge access port 34 resting on the inner back wall of the cartridge 28 is a nail stop point 50, which could be made of a composite of resins and glues or molded plastic or cork or metal, and the bottom of the cartridge 28 is sealed with the cartridge bottom plug 36.

The outer wall of the cartridge carries a square cut o-ring 30 and the saddle seal 32, which not only functions as a seal but also provides spacing between the cartridge 28 and the outer housing tube 40 in order to reduce any caking or sticking of the cartridge to the inside wall of the outer housing tube 40 and thereby ensure fluidity of movement of the cartridge inside the outer housing when a user presses down on the plunger assembly.

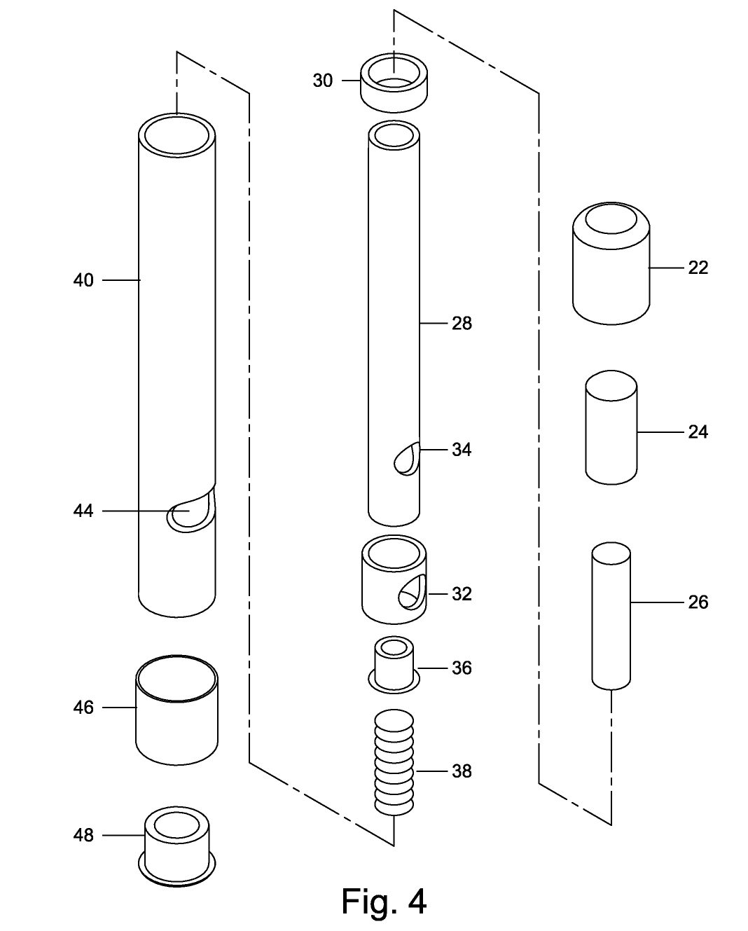

FIG. 4

Shown at the top of the article is a plunger retainer 22 that slides over the top of the plunger cover 24 and the plunger 26, finally resting on top of the outer housing tube 40. The plunger 26 is concealed by the plunger cover 24 and both the plunger 26 and plunger cover 24 are attached to the top of the inside walls of the cartridge 28 which, for example, could be made of 0.5 inch outer diameter acrylic tubing, two separate half pieces of molded plastic bonded together, an extruded piece of aluminum tubing sized to fit and move within the housing, or any suitable tubing material (or combination of materials). The cartridge 28 carries an o-ring 30. The lower portion of the cartridge is saddled with a saddle seal or bearing 32, which is centered over the cartridge access port 34. The entire cartridge 28 is sealed on the bottom with the cartridge bottom plug 36, which be made of polycarbonate plastic (or any suitable material). A biasing member 38 is disposed inside of the outer housing tube 40 in order to engage the cartridge. In the embodiment shown, the open end of the housing is sealed with an inner housing bottom plug 48, upon which the biasing member 38 rests.

Those in the art will appreciate that complementary mechanical features can be used to provide registration between the housing and cartridge.

The housing access port 44 is bored through the wall of the housing. In the embodiment shown, the housing access port is about 1″ from the bottom of the outer housing tube 40. The sliding sleeve 46 rides on the outer wall of the outer housing tube 40 and is placed atop of the housing access port 44, concealing it while in its resting position. The outer housing tube 40 is sealed on the bottom with the housing bottom plug 48. An optional magnet 54 can be attached to the inner wall of the outer housing tube 40.

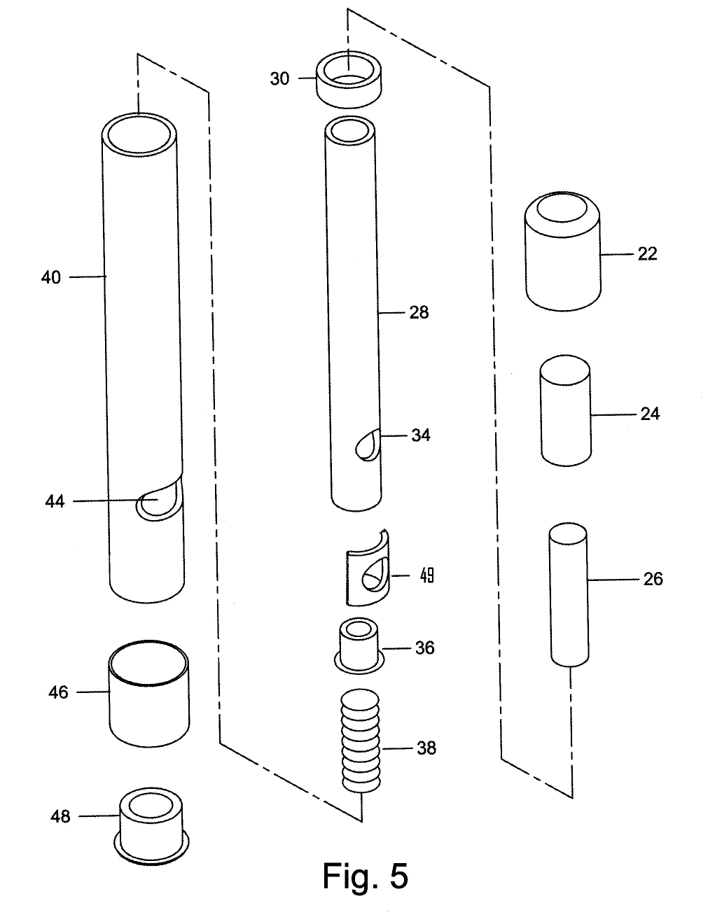

FIG. 5

This figure shows a device similar to that depicted in FIG. 4. In the embodiment shown in this Figure, bearing 32 is replaced with bearing or saddle seal 49, which has an opening or port therein. Bearing or saddle seal 49 is preferably is secured to cartridge 28 (as is also preferably the case for bearing 32 of the device shown in FIG. 4) in manner such that its opening or port aligns with the cartridge access port 34 of cartridge 28.

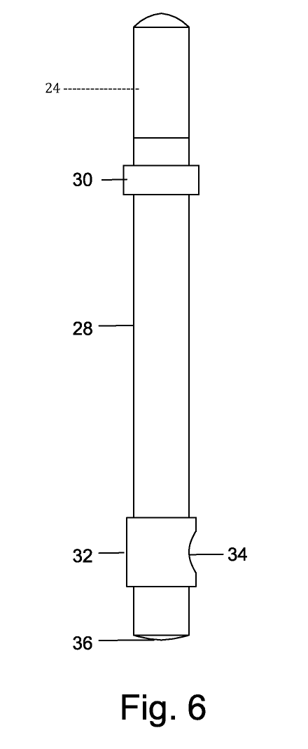

FIG. 6

The plunger cover 24 conceals the plunger 26, which is bonded to the inner wall of the cartridge 28 sealing in a powdered composition. The square cut o-ring 30 wrapped around the cartridge 28 rests above the saddle seal 32, which is positioned over the cartridge access port 34. The bottom of the cartridge 28 is sealed with the cartridge bottom plug 36.

The outer wall of the cartridge carries a square cut o-ring 30 or a Gore sealant in another embodiment. The saddle seal 32 not only functions as a seal but also acts as a spacer to provide a gap between the cartridge 28 and the inner surface of housing tube 40 in order to reduce any caking or sticking of the cartridge and providing fluidity of movement.

Operations

The manner of using a powder dispenser of the invention in its intended primary use as a styptic dispenser is as shown in FIG. 1 is straightforward. For example, a user, while cutting the toenails of a dog or other animal, inadvertently cuts into the blood supply of the nail, resulting in bleeding. The user then manually picks up the powder dispenser by the outer housing tube 40 and pushes down the sliding sleeve 46, revealing the wall of the inner cartridge 28. The user then proceeds to engage the top plunger 26 via the plunger cover 24 with his or her thumb, thus pushing down the inner cartridge 28 and bringing the cartridge access port 34 into alignment with the outer housing access port 44, thereby exposing the styptic powder in the cartridge's reservoir. The user then presses the animal's bleeding nail into the nail stop stop (50), which stops the bleeding.

The user then pulls the article away from the animal's paw while releasing his or her thumb from the plunger cover 24 and plunger 26, which returns the cartridge 28 to its starting position. In this way styptic powder can be directly delivered from the gravity fed cartridge 28, and any contaminated or used powder is fused with the nail, leaving the cartridge stocked and ready for the next needed application.

In another embodiment, the user while cooking can deliver a “pinch” of, for example, spice stored in the cartridge's reservoir. To add the spice, the user simply picks up the powder dispenser by the outer housing tube 40 and pushes down the sliding sleeve 46 revealing the wall of the inner cartridge 28. The user then proceeds to engage the top plunger 26 via the plunger cover 24 with his or her thumb thus pushing down the inner cartridge 28 and bringing the cartridge access port 34 into alignment with the outer housing access port 44, thereby exposing the powdered spice composition, the desired amount of which the user can then dispense from the reservoir before releasing his or her thumb from the plunger cover 24 to return plunger 26 of the cartridge 28 to its starting position.

In yet another embodiment, the compression spring disposed within the housing rests behind the main outer housing tube in a separate adjoining tube so as to lower the housing access port. In another embodiment the housing tube includes an access window to allow a user to visualize the cartridge in order to monitor the amount of powdered contents remaining in the cartridge's reservoir.

In still another embodiment the outer housing tube has an attached magnet. In another embodiment powder is forced through a diaphragm so as to dispense an allotted amount of powder during a particular dispensing operation. Alternatively, differing amounts of powdered contents from the reservoir can be dispensed by varying the diameter of the cartridge and/or the cartridge access port size(s).

All of the articles and methods disclosed and claimed herein can be made and executed without undue experimentation in light of the present disclosure. While the articles and methods of this invention have been described in terms of preferred embodiments, it will be apparent to those of skill in the art that variations may be applied without departing from the spirit and scope of the invention. All such variations and equivalents apparent to those skilled in the art, whether now existing or later developed, are deemed to be within the spirit and scope of the invention as defined by the appended claims.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

I claim:

1. An article for delivery of a powered composition, comprising:
a. an elongated housing that comprises a sealed end, an open end opposite the sealed end, and a housing access port disposed proximal to the sealed end that provides access to the housing's interior;
b. a cartridge disposed inside of and in moveable relation to the housing, the cartridge comprising first and second sealed ends disposed opposite each other, a reservoir for storing a powdered composition, and a cartridge access port that provides access to the reservoir, wherein the first sealed end of the cartridge faces the sealed end of the housing and the second sealed end of the cartridge faces the open end of the housing;
c. a plunger, comprising a plunger element having a flange disposed at one end, wherein the flange has a cartridge-facing surface that engages the said second sealed end of the cartridge and a retaining surface opposite the cartridge-facing surface, and an exposed surface configured for application of an actuation force by a user, wherein the plunger element extends through the open end of the housing and is retained in the housing by a plunger retainer disposed on the open end of the housing; and, optionally,
d. a biasing member disposed inside the housing and positioned to engage and return the cartridge to a resting position after an actuation force applied by a user is reduced or removed,
wherein application of an actuation force by a user causes the cartridge to move in relation to the housing and bring the cartridge and housing access ports into alignment, thereby allowing access to the reservoir.

2. An article according to claim 1 that further comprises at least one of the following:
a. an alignment system that provides complementary structures between the housing and cartridge that promotes alignment of the housing access port and cartridge access port when the cartridge moves into dispensing position;
b. a stop disposed in the reservoir opposite the cartridge access port;
c. a powdered composition in the reservoir, wherein the powdered composition optionally comprises a hemostatic compound.

3. A method of dispensing a powdered composition, comprising of and using an article according to claim 1 wherein the reservoir has been charged with the powdered composition to be dispensed, applying an actuation force to the plunger, and dispensing some or all of the powdered composition from the reservoir.
